(19) United States
(12) Patent Application Publication
(10) Pub. No.: US 2025/0331750 A1
(43) Pub. Date: Oct. 30, 2025

(54) SEPARATING GEL FOR BLOOD COLLECTION TUBES

(71) Applicant: Greiner Bio-One GmbH, Kremsmunster (AT)

(72) Inventors: Christian Moser, Schwanenstadt (AT); Manuel Schulz, Alkoven (AT); Bernhard Schwabegger, Wartberg/Aist (AT)

(21) Appl. No.: 19/192,290

(22) Filed: Apr. 28, 2025

(30) Foreign Application Priority Data
Apr. 29, 2024 (AT) A 50358/2024

(51) Int. Cl. A61B 5/15; C08G 18/67

(52) U.S. Cl. CPC A61B 5/150755; C08G 18/672

(57) ABSTRACT

A separator gel for blood collection tubes for the separation of blood serum or blood plasma from blood cells is disclosed. The separator gel includes an acrylate copolymer, silica and silicone oil and/or at least one polyalkylene glycol. The separator gel contains ≤1000 ppm of solvent. A method for producing the separator gel, a blood collection tube with the separator gel, as well as a method for producing the acrylate copolymer are disclosed. For the purification of the acrylate copolymer at least one solvent is separated by multistage distillation, such that a residual solvent content of ≤1000 ppm is achieved.

### CROSS-REFERENCE TO RELATED APPLICATIONS

This application claims priority to Austrian (AT) patent application no. A 50358/2024 filed on Apr. 29, 2024, the contents of which are hereby incorporated by reference in its entirety.

### TECHNICAL FIELD

The invention relates to a separator gel for blood collection tubes for the separation of blood serum or blood plasma from blood cells comprising an acrylate copolymer, silica and silicone oil and/or at least one polyalkylene glycol, a method for the production thereof, a blood collection tube for the separation of blood serum or blood plasma with a separator gel, a method for separating blood serum or blood plasma with a blood collection tube, as well as a method for producing the acrylate copolymer for a separator gel for blood collection tubes.

### BACKGROUND

Clinical analysis technologies for detecting biochemical substances require the separation of whole blood into its component parts, i.e. serum or plasma, and blood cells. The separated portion ought to be as free of blood cells as possible so as not to impair the clinical analyses.

Blood collection tubes with separator gel are frequently used for obtaining serum or plasma for laboratory analyses. The separator gel at the bottom of the tube has a lower density than the coagulation proteins and blood cells aggregated during clotting, and during centrifugation passes between the blood cells and serum since its physical density is between the two fractions. A separation layer thus forms which prevents a contamination of the diagnostic sample, in particular of the serum with the blood cell components, and, for example, also prevents the breakdown of glucose by blood cells.

To determine clinical parameters such as glucose, potassium and phosphorus, the serum has to be separated rapidly from the blood cells as the measurement values would otherwise be distorted.

Due to the diffusion barrier formed by the separator gel, it is still possible to detect clinical chemistry analytes such as steroids, hormones, vitamins and medications even after a longer period of cooled storage.

U.S. Pat. No. 5,438,000 describes a serum separating agent with an excellent balance of flow and specific gravity properties and excellent storage stability. The serum separating agent has at 20° C. a specific gravity of 1.035 to 1.065, a viscosity of 100 to 400 Pa·s and a yield stress of 100 bis 400 dyne/cm² and comprises (A) 100 parts by weight of a polymer with a specific weight at 20° C. of 0.94 to 1.06 and a viscosity of 10 to 140 Pa·s, derived from an alkyl acrylate or alkyl methacrylate monomer; (B) 0.5 to 10 parts by weight of at least one component, selected from the group consisting of silicon dioxide and bentonite; and (C) 0.01 to 2 parts by weight of at least one surfactant, selected from the group consisting of: (C-1) fluorocarbon-based surfactants; (C-2) polyester modified alkylpolysiloxane-based surfactants; and (C-3) polyether modified alkylpolysiloxane-based surfactants and, optionally, (D) 0.01 to 1 parts by weight of at least one component selected from the group consisting of titanium dioxide and calcium carbonate; and (E) 0.02 to 1 parts by weight of a titanium-based coupling agent, based on 100 parts by weight of polymer (A).

A composition for a separator gel for separating blood serum or blood plasma in a blood collection container is known from EP3734273A1. The composition comprises a (meth)acrylic acid ester-based polymer, silicon dioxide and a silicone oil, wherein the polymer has fluidity at room temperature and has a molecular weight of 15,000 or more and 100,000 or less.

Blood collection tubes with separator gel known in the art have a residual solvent content of toluene or N-Methyl-2-pyrrolidone (NMP) of greater than 1,000 ppm. These substances are classified as hazardous substances according to Directive (EC) No. 1272/2008.

In one blood collection tube with separator gel known in the art, toluene is declared as a hazardous substance with a content over 0.1%, for instance.

A further blood collection tube with separator gel on the market contains N-Methyl-2-pyrrolidone (NMP), a hazardous substance classified as an SVHC substance, in a quantity of almost 3,000 ppm.

Such residual solvent contents are generally achieved through solvent separation by means of a conventional distillation process.

The object of the present invention is to overcome the disadvantages of the prior art and to provide a composition and a method by means of which a user is able to perform a simple separation of blood serum or blood plasma from blood cells.

### SUMMARY

This object is solved by a separator gel, an acrylate copolymer, a blood collection tube and a method for producing the separator gel and the acrylate copolymer according to the claims.

The separator gel for the blood collection tube for the separation of blood serum or blood plasma from blood cells comprising at least an acrylate copolymer, silica and silicone oil and/or at least one polyalkylene glycol has ≤1000 ppm, preferably ≤300 ppm of solvent, whereby the separator gel is not classified as a hazardous substance and thus the safety measures and safety provisions for the product, in particular for blood collection tubes where the separator gel according to the invention is used, can be reduced.

In addition, a high purity of the components is achieved by using the separator gel according to the invention in a blood collection tube, which minimizes the risk of contamination of the blood sample.

This also results in a better sample quality, which can have an effect on subsequent analytic use.

The separator gel according to the invention and the method for producing the same are very sustainable and safe.

For the polymerization of the acrylate copolymer for producing a polymer component, it is intended to use at least two monomers, in particular n-butyl acrylate and 2-ethylhexyl acrylate, whereby the selection of the components, in particular of the monomers, enables an efficient production of the separator gel.

The monomer ratio of n-butyl acrylate to 2-ethylhexyl acrylate for the production of the polymer components is preferably 2:1 to 9:1, in particular 3:1 to 5:1, preferably 4:1, as the required density of the polymer can thereby be optimally configured.

The acrylate copolymer of the separator gel preferably has a density of 1.010 g/cm³ to 1.040 g/cm³, preferably 1.025 g/cm³ to 1.035 g/cm³, in particular between 1.030 g/cm³ to 1.033 g/cm³, at 20° C., whereby a separator gel can be produced which forms a stable separation layer with sufficient rigidity even under temperature fluctuations.

The acrylate copolymer of the separator gel has a viscosity of 60 Pa·s to 180 Pa·s, preferably 70 Pa·s to 130 Pa·s, in particular 90 Pa·s to 110 Pa·s, at 20° C., whereby a satisfactory fluidity of the separator gel can be achieved at room temperature.

According to a preferred embodiment, the acrylate copolymer contains ≤1000 ppm, preferably ≤300 ppm, of solvent, which results in a reduced chemical influence of the residual solvent content on the blood analysis compared to blood collection tubes available on the market. The sample quality and the analytic stability are thus increased and analysis results with better reproducibility can also be achieved.

The acrylate copolymer preferably has a residual content of n-butyl acrylate monomers of ≤50 ppm, in particular ≤20 ppm, and/or a residual content of 2-ethylhexyl acrylate monomers of ≤200 ppm, preferably ≤100 ppm, in particular ≤80 ppm, whereby the risks of sample contamination can be reduced and the quality of the blood sample and the clinical parameter analytics performed therewith are improved.

The processing problems during sterilization are also reduced due to the lower monomer content.

In order to prevent bacterial infection of the patient and the sample by the blood collection tube, the blood collection tube is sterilized during the production process using electron irradiation, gamma rays or x-rays to fulfil ISO standards. The robustness of the separator gel in the subsequent sterilization process is increased by the high purity, as monomeric contaminations would lead to an undesired cross-linking of the separator gel. That cross-linking could lead to an undesired change of the thixotropic properties.

Silicone oil and/or at least one polyalkylene glycol is contained in the separator gel in a total quantity of 0.01 wt. % to 1 wt. %, preferably 0.05 wt. % to 0.75 wt. %, in particular 0.1 wt. % to 0.5 wt. %, whereby the required thixotropy of the separator gel and at the same time a good dispersibility of the silica are achieved. Phase dissolution, where separated components of low viscosity flow out of the phase during storage, can also be prevented.

Pyrogenic untreated hydrophilic and/or modified hydrophobic silica is preferably used as silica, in particular in a quantity of 0.5 wt. % to 5 wt. %, preferably 1 wt. % to 4 wt. %, in particular 2 wt. % to 3 wt. %.

A further embodiment of the separator gel contains titanium dioxide, in particular in a quantity of 0.001 wt. % to 0.1 wt. %, preferably 0.005 wt. % to 0.08 wt. %, in particular 0.01 wt. % to 0.05 wt. %, whereby together with the silica the density of the separator gel can be configured.

Advantageously, the separator gel has a density of 1.038 g/cm³ to 1.058 g/cm³, preferably 1.040 g/cm³ to 1.050 g/cm³, in particular 1.044 g/cm³ to 1.048 g/cm³, at 20° C., whereby a stable separation layer with sufficient rigidity even under temperature fluctuations can be formed.

The separator gel has a viscosity of 200 Pa·s to 520 Pas, preferably 220 Pa·s to 280 Pa·s, at 20° C., whereby the formation of a stable and rigid separation layer for storing the blood sample separated in phases is possible.

It is advantageous that the separator gel has a thixotropic index between 1.2 and 2.2, in particular between 1.2 and 1.7, preferably between 1.3 and 1.6, as shear liquefaction of the separator gel can thus be ensured and the formation of a gel separation layer occurs under corresponding centrifugation conditions.

The method for producing the separator gel according to the invention for blood collection tubes for the separation of blood serum or blood plasma comprising at least an acrylate copolymer, silica and silicone oil and/or at least one polyalkylene glycol provides that the acrylate copolymer, silica and silicone oil and/or at least one polyalkylene glycol and where applicable titanium dioxide are mixed, thus providing a sustainable and safe production process.

It has proven advantageous that for producing the polymer component of the acrylate copolymer at least n-butyl acrylate is used in a quantity of 30 wt. % to 50 wt. %, preferably 35 wt. % to 45 wt. %, in particular 38 wt. % to 42 wt. %, and 2-ethylhexyl acrylate is used in a quantity of 2 wt. % to 20 wt. %, preferably 5 wt. % to 15 wt. %, in particular 8 wt. % to 12 wt. %, whereby the density of the polymer can be configured.

According to a preferred embodiment, an aromatic solvent, in particular toluene or xylene, is used as a solvent for the production of the polymer component, preferably in a quantity of 30 wt. % to 70 wt. %, preferably 40 wt. % to 60 wt. %, in particular 45 wt. % to 55 wt. %, having a lower toxicity compared to the benzene frequently used as a solvent.

As an initiator for the polymerization of the at least two monomers, an organic peroxide, in particular 1,1,3,3-tetramethylbutylperoxy-2-ethylhexanoate, is used for the production of the polymer component, preferably in a quantity of 0.05 wt. % to 5 wt. %, preferably 0.1 wt. % to 1 wt. %, in particular 0.3 wt. % to 0.6 wt. %, where the viscosity of the polymer can be configured through the concentration of the initiator. Compared to the frequently used initiator azobisisobutyronitrile (AIBN), this initiator has the advantage that no toxic tetramethylsuccinonitrile (TMSN) remains in the separator gel as a residue.

A further embodiment of the method provides that the untreated hydrophilic silica is pre-conditioned with a drying step. A low moisture content of the silica and thus a constant quality of the separator gel produced with it is thereby ensured. The thixotropic properties that arise due to the interaction of silica and silicone oil and/or at least one polyalkylene glycol are thereby also improved.

A blood collection tube for the separation of blood serum or blood plasma from blood cells with a separator gel of at least acrylate copolymer, silica and silicone oil and/or at least one polyalkylene glycol, wherein the separator gel contains ≤1000 ppm, preferably ≤300 ppm of solvent, has the advantage that it is not classified as a hazardous substance and thus no occupational safety and health restrictions are necessary.

A blood collection tube with a separator gel with a low residual solvent content also significantly increases the stability of the analytes for the period between centrifugation and analysis. This stability gain through the gel barrier is also an advantage during transport and storage of the blood in blood collection tubes. Moreover, this stable barrier between serum and coagulum also enables better analyte stability.

The workflow from taking blood until analysis can also be optimized by a blood collection tube according to the invention. Short centrifugation times are thus possible, the sample processing and archiving can occur in the primary tube, and there is no risk of mix-up from the use of a secondary tube.

Preferably, the acrylate copolymer according to the invention for a separator gel for blood collection tubes comprises at least two monomers and a solvent, wherein ≤50 ppm, in particular ≤20 ppm of a first monomer, and ≤200 ppm, preferably ≤100 ppm, in particular ≤80 ppm of a second monomer, as well as ≤ 1000 ppm, in particular ≤300 ppm of solvent are contained, wherein due to the low residual monomer content and thus the high purity of the acrylate copolymer for a separator gel, the risks of sample contamination with monomers or solvent residues are minimized and the quality of the blood sample and the clinical parameter analytics performed therewith are improved.

A residual solvent content of ≤1000 ppm, in particular ≤300 ppm, is achieved by the method according to the invention for producing an acrylate copolymer for a separator gel for blood collection tubes through radical solvent polymerization in feed procedure from a polymer solution, wherein for the purification of the acrylate copolymer, at least the solvent is separated by multistage distillation. The distribution of hazardous substances onto the market is thereby reduced because they are separated in the process and fed back for re-use.

By means of the method according to the invention, residual monomers are also separated from the polymer solution of the acrylate copolymer by multistage distillation, such that ≤50 ppm, in particular ≤20 ppm of a first monomer, in particular n-butyl acrylate, and ≤200 ppm, preferably ≤100 ppm, in particular ≤80 ppm of a second monomer, in particular 2-ethylhexyl acrylate, are achieved, whereby a highly pure separator gel can be produced which enables a reproducible analysis of blood samples, in particular of analytes from the blood serum or blood plasma.

Preferably, the distillations are performed continuously with wiped film rotation evaporators, whereby lower values of solvent in the acrylate copolymer for the production of a separator gel can be achieved.

According to a further embodiment of the method, the polymer discharged from the wiped film rotation evaporator is fed into a flash evaporation stage, and where applicable is subjected beforehand to interim heating, whereby a better removal of the still remaining solvent and the residual monomers can be achieved.

Preferably, the separation of the solvent and where applicable the residual monomers is performed in a vacuum, as better results can thereby be achieved.

In a subsequent short-path evaporation stage, the solvent and where applicable the residual monomers are distilled off to their desired final content and thus an acrylate copolymer optimized for further processing is provided.

According to a further embodiment of the method, the solvent and where applicable the residual monomers are frozen out and subsequently discharged in the downstream cold trap system, whereby they can be fed back for re-use.

### BRIEF DESCRIPTION OF THE DRAWINGS

For a better understanding of the invention, it is explained in more detail with reference to the following figures.

These show in significantly simplified, schematic representation:

A flow diagram for the production and purification of the acrylate copolymer as well as for the production of the separator gel.

### DETAILED DESCRIPTION

The invention comprises a separator gel on the basis of a highly purified acrylate copolymer which, through the addition of a rheological additive, in particular a modified silicone oil and/or at least one polyalkylene glycol, and silica, displays the required rheological properties in relation to shear liquefaction and at the same time also has the ideal density for achieving a separation of blood serum or blood plasma from blood cells.

Shear forces act upon the separator gel during the centrifugation of a blood sample that lead to a liquefaction. In addition, lifting forces during the centrifugation act upon the separator gel, which detaches itself from the blood collection tube base and rises. In accordance with its density, the separator gel collects in the region between the blood cells and the remainder, in particular the blood serum or blood plasma, where it forms a stable separation layer between the phases.

The configuration of the correct density and the adherence to strict specification limits is, along with the thixotropic properties of the separator gel, essential for correct functioning during centrifugation and the formation of the separation layer. The high purity of the separator gel minimizes the risk of sample contamination and improves the quality of the blood sample and the clinical parameter analytics performed therewith, and thereafter also the safety of the material in further processing and use.

The present invention comprises a separator gel for blood collection tubes for the separation of blood serum or blood plasma from blood cells comprising an acrylate copolymer, silica and silicone oil and/or at least one polyalkylene glycol, wherein the separator gel contains ≤1000 ppm, preferably ≤300 ppm, of solvent.

The separator gel according to the invention is used for the separation of blood serum or blood plasma from the other component parts of the blood and forms a sealed separation layer and diffusion barrier such that even after longer storage no cell content can enter the serum or plasma. The disruptive influence of the cells, e.g. hemolysis, glucose breakdown or potassium release, is thereby prevented.

Hemolysis destroys the cell membrane of the erythrocytes, whereby intracellular component parts can enter the serum or plasma. If serum or plasma is not separated from the cells, either by a separator gel or following centrifugation by pipetting into a secondary receptacle, substances from the cells pass through into the plasma or serum. The cell wall is not actually destroyed during this process as occurs during hemolysis, but the effects on the sample are similar. The result is, for instance, increased LDH and potassium values. Blood sugar is broken down by glycolysis. The cells thus also absorb glucose from the serum or plasma in vitro. The blood sugar level thereby continually changes over time. If serum or plasma is not separated from the cells, this process can already lead to significant changes after two hours.

A separator gel according to the present invention is not categorized as a hazardous substance and reduces work-related health risks, thus posing less risk for staff in the healthcare sector and providing significant simplification for employers.

The component parts of the separator gel according to the invention comprise at least an acrylate copolymer, silica and silicone oil and/or at least one polyalkylene glycol. The separator gel according to the invention can also consist of at least an acrylate copolymer, silica as well as a silicone oil and/or at least one polyalkylene glycol.

According to the invention, silicon dioxide and silica are used equivalently and comprise both pyrogenic and precipitated silicon dioxide or silica.

The separator gel for the separation of blood serum or blood plasma according to the present invention comprises an acrylate copolymer which is formed by the polymerization of at least two different polymerizable monomers.

According to a preferred embodiment, at least two monomers, in particular n-butyl acrylate (NBA) and 2-ethylhexyl acrylate (EHA), at least one solvent and at least one initiator are used for the production of the polymer components of the acrylate copolymer.

According to the invention, polymer component is understood as the polymer solution in the production process for producing the acrylate copolymer and polymer or copolymer is understood as the purified polymer largely free of monomers and solvent.

The acrylate copolymer is preferably produced through radical solvent polymerization, preferably using the radical polymerization method according to the invention.

Polymerizable monomers that can be used for the production of the acrylate copolymer of the separator gel according to the invention are what are known as alkyl acrylates or alkyl methacrylates. Examples of such monomers are methyl(meth)acrylate, ethyl(meth)acrylate, n-butyl(meth)acrylate, i-butyl (meth)acrylate, 2-ethylhexyl (meth)acrylate, lauryl (meth)acrylate, tridecyl (meth)acrylate and stearyl (meth)acrylate. Among these, n-butyl(meth)acrylate or 2-ethylhexyl (meth)acrylate or copolymers obtained through the combined use of those monomers are preferred, as these polymers have a suitable viscosity, are easy to handle, and are able to easily disperse silicon dioxide in order to configure the desired density or viscosity.

Furthermore, any copolymerizable monomer can additionally be used in combination therewith. Typical examples of such copolymerizable monomers are ethylene glycol diacrylate, propylene glycol diacrylate, neopentyl glycol diacrylate, 1,6-hexanediol diacrylate, diethylene glycol diacrylate, triethylene glycol diacrylate, tetraethylene glycol diacrylate, ethylene glycol dimethacrylate, propylene glycol dimethacrylate, neopentyl glycol dimethacrylate, 1,6-hexanediol dimethacrylate, diethylene glycol dimethacrylate, 1,2,3-propanetriol di(meth)acrylate, divinylbenzene and the like.

Further examples of the monomer include a radically polymerizable monomer which is capable of radical copolymerization with the (meth)acrylic acid ester monomer.

Examples of the radically polymerizable monomer also include aromatic vinyl monomers, vinyl esters, vinyl ethers, vinylpyrrolidone and (meth)allyl ethers.

For the radically polymerizable monomer, it is possible to use just one radically polymerizable monomer or a combination of two or more radically polymerizable monomers.

Examples of the aromatic vinyl monomers include styrene, α-methylstyrene, p-methylstyrene, α-methyl-p-methylstyrene, p-methoxystyrene, o-methoxystyrene, 2,4-dimethylstyrene, chlorosytrene and bromostyrene.

Examples of vinyl esters include (meth)acrylate, maleic anhydride, fumarate, (meth)acrylamide, dialkyl-(meth)acrylamide and vinyl acetate. The radically polymerizable monomer is preferably the aromatic vinyl monomer.

The highly purified acrylate copolymer according to the invention is obtained through polymerization of an alkyl acrylate and/or alkyl methacrylate.

According to a preferred embodiment, the monomer ratio of n-butyl acrylate to 2-ethylhexyl acrylate is 2:1 to 9:1, in particular 3:1 to 5:1, preferably 4:1, for the production of the polymer component for a separator gel. The ratio of the monomers allows for the density of the acrylate copolymer to be configured.

For the production of the polymer component at least n-butyl acrylate is used in a quantity of 30 wt. % to 50 wt. %, preferably 35 wt. % to 45 wt. %, in particular 38 wt. % to 42 wt. %, and 2-ethylhexyl acrylate is used in a quantity of 2 wt. % to 20 wt. %, preferably 5 wt. % to 15 wt. %, in particular 8 wt. % to 12 wt. %.

The percentage of the overall mass of the polymer solution for the production of the acrylate copolymer for the separator gel is thus preferably approximately 50 wt. % solvent, 40 wt. % n-butyl acrylate and 10 wt. % 2-ethylhexyl acrylate and an initiator in a quantity of less than 1 wt. %.

As a solvent, an aromatic solvent, preferably toluene or xylene, is used for the production of the polymer component for the production of the acrylate copolymer for the separator gel.

According to a preferred embodiment, the at least one solvent is preferably used in a quantity of 30 wt. % to 70 wt. %, preferably 40 wt. % to 60 wt. %, in particular 45 wt. % to 55 wt. %, for the production of the polymer component.

Toluene is a CMR substance. CMR substances are classified as being carcinogenic, mutagenic and reprotoxic. Even if CMR substances and preparations only have low or no acutely noticeable negative properties, prolonged contact with them can have an unhealthy and dangerous effect on the organism without it being perceived as dangerous by the affected person.

Because toluene is a CMR substance, the product safety of the separator gel is significantly improved by the low solvent content. The separator gel according to the invention is not classified as a hazardous substance, resulting in advantages with regard to transport and occupational safety and health amongst others.

According to a preferred embodiment, toluene is used as a solvent for the production of the polymer component of the separator gel. According to an alternative embodiment, xylene can also be used as a solvent for the production of the polymer component of the separator gel. Xylene is not classified as a CMR substance and is categorized neither as mutagenic, carcinogenic nor reprotoxic.

As an initiator for the polymerization of the at least two monomers for the production of the polymer component, an organic peroxide, in particular 1,1,3,3-tetramethylbutylperoxy-2-ethylhexanoate, is used for the production of the polymer component

The polymerization of the at least two monomers is preferably performed with an organic peroxide as a polymerization initiator. For example, Trigonox® 421 can be used as an organic peroxide, whereby unlike with the use of azobisisobutyronitrile (AIBN) as a polymerization initiator no toxic tetramethylsuccinonitrile (TMSN) is formed as a degradation product.

The at least two monomers can, however, also be polymerized with an azo-based polymerization initiator.

The acrylate copolymer for the production of the separator gel has a density of 1.010 g/cm³ to 1.040 g/cm³, preferably 1.025 g/cm³ to 1.035 g/cm³, in particular between 1.030 g/cm³ to 1.033 g/cm³, determined at a temperature of 20° C. The density of the acrylate copolymer is determined at a temperature of 20° C. using a DMA4500M oscillating U-tube density meter (Anton Paar) or a gas pycnometer (Pycnomatic ATC).

The viscosity of the acrylate copolymer is in a range from 60 Pas to 180 Pa·s, preferably 70 Pa·s to 130 Pas, in particular 90 Pas to 110 Pa·s, determined at a temperature of 20° C. The viscosity is measured with a Brookfield viscometer with cone-plate measuring system (CPA-52Z measuring cone, 3° angle) at a shear rate of 5/sec and a temperature of 20° C.

After purification, the acrylate copolymer has ≤1000 ppm, preferably ≤300 ppm, of solvent. Gas chromatography measurements of different batches of purified acrylate copolymer show that, insofar as toluene is used as a solvent during polymerization, even less than 50 ppm toluene, in particular between 10 ppm and 50 ppm toluene, are contained.

After purification, the acrylate copolymer has a residual content of ≤50 ppm, in particular ≤20 ppm, of n-butyl acrylate. Here again, gas chromatography measurement results of different batches of purified acrylate copolymer show less than 5 ppm of the n-butyl acrylate monomer.

The separator gel according to the invention contains a purified acrylate copolymer with a residual content of 2-ethylhexyl acrylate monomer of ≤200 ppm, preferably ≤100 ppm, in particular ≤80 ppm. Gas chromatography measurements show that after purification of the acrylate copolymer, less than 20 ppm of 2-ethylhexyl acrylate monomer are contained.

The following Table 1 shows the specification of a preferred embodiment of a purified acrylate copolymer according to the invention, which is used for the production of the separator gel.

TABLE 1

| Parameter | | |
|---|---|---|
| Viscosity at 20° C. | 90-110 | Pa · s |
| Density at 20° C. | 1.030-1.033 | g/cm³ |
| Toluene | ≤300 | ppm |
| n-butyl acrylate | ≤20 | ppm |
| 2-ethylhexyl acrylate | ≤80 | ppm |

The quantities given in the following in connection to the composition of the separator gel are related to 100 wt. % of the composition of the separator gel for the separation of blood serum or blood plasma.

According to the invention, in addition to the acrylate copolymer the separator gel also contains silicone oil and/or at least one polyalkylene glycol. The total quantity of silicone oil and/or the at least one polyalkylene glycol is preferably 0.01 wt. % to 1 wt. %, preferably 0.05 wt. % to 0.75 wt. %, in particular 0.1 wt. % to 0.5 wt. %, related to 100 wt. % of the composition for the separation of blood serum or blood plasma. If the total quantity of silicone oil and/or the at least one polyalkylene glycol deviates from the limits defined above, it can lead to the degradation of the phase separation and the mixing of phases of differing density.

Preferably, the separator gel contains silicone oil on the basis of polyether-modified polysiloxane as a rheological additive. Silicone oil serves to configure the viscosity and the required thixotropy can thereby be achieved in combination with the silica.

Examples of possible silicone oils include dimethyl silicone oils, methyl phenyl silicone oils, methyl hydrogen silicone oils, alkyl-modified silicone oils, aralkyl-modified silicone oils, fluorine-modified silicone oils, amino-modified silicone oils, epoxide-modified silicone oils, phenol-modified silicone oils, carboxy-modified silicone oils, methacrylate-modified silicone oils and alkoxy-modified silicone oils.

It is possible to use just one silicone oil, or two or more silicone oils can also be used in combination.

As an alternative or in combination with silicone oil, at least one polyalkylene glycol with a number average molecular mass (Mn) from a range of 100 to 10000 Da, preferably 200 to 5000 Da, in particular 400 to 4000 Da can be contained in the separator gel as a rheological additive. Possible polyalkylene glycols are polyethylene glycol (PEG) (R═CH2-CH2-O) such as polyethylene glycol-400, propylene glycol (PPG) (R═CH2-CH2-(CH3)-O) such as polypropylene glycol-400 and polypropylene glycol-1000, block copolymers and statistical copolymers of ethylene oxide and propylene oxide units such as poly(propylene glycol)-block-poly(ethylene glycol)-block-poly(propylene glycol), poly(ethylene glycol)-block-poly (propylene glycol)-block-poly(ethylene glycol). Furthermore, polyalkylene glycols with OH end groups that can be fully or partially substituted by alcohols, such as polypropylene glycol monobutyl ether, can be used.

The separator gel according to the invention also contains silicon dioxide. Silicon dioxide is preferably contained in the form of synthetic, amorphous silica, in particular in a quantity of 0.5 wt. % to 5 wt. %, preferably 1 wt. % to 4 wt. %, in particular 2 wt. % to 3 wt.

The silica used can be either an untreated hydrophilic form or a modified hydrophobic silica, or a mixture of the two.

Modified silicas contain covalently bonded organic groups on their surface in order to achieve hydrophobic properties. The production is often performed by reacting the freely accessible silanol groups (Si—OH) on the surface of the untreated silica with silanes, silazanes or siloxanes.

Examples of these organic compounds include dimethyldichlorosilane, octamethyltetrasiloxane, polydimethylsiloxane, methacrylsilane, octylsilane, hexamethyldisilazane, hexadecylsilane.

Silica is used for the gel network formation and as an agent to control density. In combination with the silicone oil and/or at least one polyalkylene glycol, the silica can be used to configure both the thixotropy and the density of the separator gel.

The average equivalent diameter of the particles of the silica is preferably in a range of 10 nm to 100 nm, in particular 5 nm to 30 nm. The particle diameter is determined by means of laser diffraction.

The separator gel can contain a further inorganic powder, in particular titanium dioxide, in particular in a quantity of 0.001 wt. % to 0.1 wt. %, preferably 0.005 wt. % to 0.08 wt. %, in particular 0.01 wt. % to 0.05 wt. %.

Titanium dioxide and silica are trapped in the gel as non-soluble particles and are used to exactly configure the density of the separator gel.

Yet further inorganic powders such as zinc oxide powder, aluminum oxide powder, fine glass powder, talcum powder, kaolin powder, bentonite powder and zirconium powder can be contained in the separator gel.

The separator gel according to the present invention can contain other components than the components described above as long as the effects of the present invention are not impaired, in particular that the separation property of the separator gel is still guaranteed and the absence of a hazardous substance is maintained.

For example, an antioxidant and a dye can be contained as further components of the present invention. For each of the other components, it is possible for the separator gel to contain just one component or two or more components in combination.

The separator gel according to the invention has a density of 1.038 g/cm³ to 1.058 g/cm³, preferably 1.040 g/cm³ to 1.050 g/cm³, in particular 1.044 g/cm³ to 1.048 g/cm³, at 20° C. The density of the separator gel in the selected range enables the formation of a stable separating wall with sufficient rigidity between the blood serum or blood plasma and the remaining component parts of the blood even with low quantities of blood cells or blood cell component parts, low temperatures and/or when using a low centrifugal force.

The density of the separator gel is determined at a temperature of 20° C. using a DMA4500M oscillating U-tube density meter (Anton Paar) or a gas pycnometer (Pycnomatic ATC).

The viscosity of the separator gel is in a range of 200 Pa·s to 520 Pa·s, preferably 220 Pa·s to 280 Pa·s, at a temperature of 20° C. The viscosity is measured with a Brookfield viscometer with a cone-plate measuring system (CPA-52Z measuring cone, 3° angle) at a shear rate of 1/sec and a temperature of 20° C. until a stable measurement value is obtained.

The thixotropic index (TI) of the separator gel according to the invention is between 1.2 and 2.2, in particular between 1.2 and 1.7, preferably between 1.3 and 1.6.

The thixotropic index is the quotient of two viscosity measurement values at different shear rates. The viscosity is thereby measured with a Brookfield viscometer with a cone-plate measuring system (CPA-52Z measuring cone, 3° angle) at a temperature of 20° C. The measurement is first carried out at a shear rate of 1/sec until a stable measurement value is obtained, and then at a shear rate of 5/sec until a stable measurement is obtained. The calculation of the thixotropic index is performed according to Formula 1.

$$TI = \frac{Viscosity at shear rate 1 / sec}{Viscosity at shear rate 5 / sec} \qquad Formula 1$$

Gas chromatography measurements of the separator gel show a low quantity of toluene solvent and low quantities of the n-butyl acrylate and 2-ethylhexyl acrylate monomers. The measurement is performed by means of headspace gas chromatography with a flame ionization detector. When using a purified acrylate copolymer for its production, the separator gel according to the invention has a residual content of n-butyl acrylate monomer of ≤50 ppm, in particular ≤20 ppm, and a residual content of 2-ethylhexyl acrylate monomer of ≤200 ppm, preferably ≤100 ppm, in particular ≤80 ppm.

According to particularly preferred embodiments, the separator gel has a residual content of ≤5 ppm of n-butyl acrylate monomer and ≤20 ppm of 2-ethylhexyl acrylate monomer and at the same time a residual toluene content of ≤50 ppm.

The following Table 2 shows the specification of a possible embodiment variation of the separator gel according to the invention.

TABLE 2

| Parameter | | |
|---|---|---|
| Viscosity at 20° C. | 200-520 | Pa · s |
| Thixotropic index | 1.2-2.2 | |
| Density at 20° C. | 1.038-1.058 | g/cm³ |
| Toluene (GC) | ≤300 | ppm |
| n-butyl acrylate (GC) | ≤20 | ppm |
| 2-ethylhexyl acrylate (GC) | ≤80 | ppm |

The production of the separator gel according to the invention for blood collection tubes for the separation of blood serum or blood plasma can, for example, be performed by mixing the purified acrylate copolymer according to the invention, silica and silicone oil and/or at least one polyalkylene glycol and where applicable titanium dioxide and where applicable other optional components.

According to a preferred embodiment, the untreated hydrophilic silica is pre-conditioned with a drying step. The drying can be performed by temperature increase, pressure reduction (vacuum) or through-flow with dry gas (carrier gas). The use of a combination of two or all three of these methods increases the efficiency of the drying with regard to the duration and residual moisture achievable. A carrier gas (dry compressed air) is flowed through the silica in a heated receptacle at 60° C. in a vacuum. To monitor the progress of the drying, the moisture of the waste gas is measured and ceased when a value of <1% relative humidity is measured in the waste gas flow.

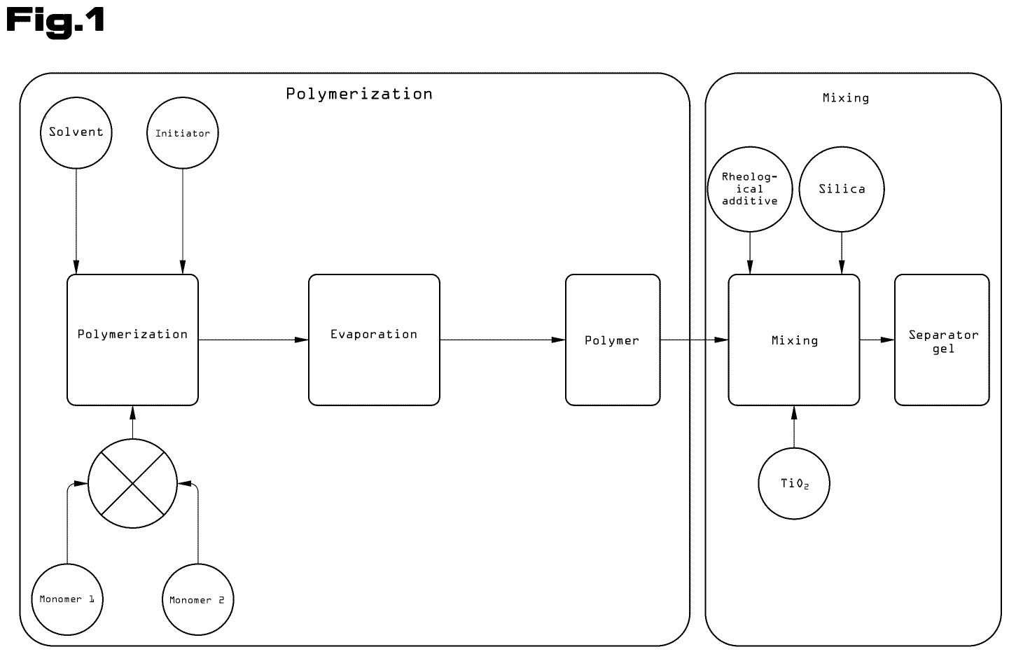

FIG. 1 shows a flow diagram of the production method of the separator gel according to the invention comprising the polymerization and purification of the acrylate copolymer and the mixing of the components of the separator gel according to the invention.

For the production of the polymer component of the acrylate copolymer, at least two monomers, preferably the two monomers n-butyl acrylate and 2-ethylhexyl acrylate, are mixed and the initiator is subsequently fed into the provided boiling solvent in feed procedure and supplied to the reactor for radical solvent polymerization.

In order to separate the solvent and the monomers after polymerization from the polymer component of the acrylate copolymer, evaporation is performed in multiple stages, wherein preferably a wiped film distillation is performed, followed by a flash evaporation stage and a short-path evaporation stage.

The method for the production of the separator gel can be carried out using a known mixer, for instance a planetary mixer, a ball mill or a dispersion machine.

According to the method for the production of a separator gel for blood collection tubes for the separation of blood serum or blood plasma comprising an acrylate copolymer, silica and silicone oil and/or at least one polyalkylene glycol, the acrylate copolymer, silica and silicone oil and/or at least one polyalkylene glycol and where applicable titanium dioxide are mixed in a vacuum planetary dissolver at 60° C., with a negative pressure of ≤40 mbar for approximately 60 minutes. The circumferential speed of the dissolver disc is preferably approximately 18 m/s to 30 m/s. A higher mixing speed has proven advantageous in the mixing process. An established method for the production of the separator gel according to the invention can thereby be used.

In order to obtain the required thixotropy and achieve a satisfactory dispersibility of the silica, the acrylate copolymer and the silicone oil and/or the at least one polyalkylene glycol are first mixed and the silica and where applicable the titanium dioxide are subsequently mixed in and where necessary further components added to the resulting mixture. According to a preferred embodiment, the silica is pre-conditioned by drying before it is added to the acrylate copolymer silicone oil mixture and/or acrylate copolymer-polyalkylene glycol mixture.

The separator gel according to the invention is contained in a blood collection tube for the separation of blood serum or blood plasma. According to the present invention, a tube comprises at least the separator gel according to the invention, wherein the separator gel is contained in the blood collection tube. The separator gel is preferably contained in the closed end of the blood collection tube.

Blood clotting is activated to obtain blood serum, e.g. by silica particles on the blood collection tube inner wall or with additional thrombin in serum tubes.

VACUETTE® CAT serum clot activator tubes are coated with micronized silica particles that activate clotting and which are suspended in the blood sample by turning the filled blood collection tube.

VACUETTE® CAT Serum Fast Separator tubes contain a separator gel in the blood collection tube base. In contrast to conventional serum tubes, the Serum Fast tube contains additional thrombin to accelerate the clotting process.

Serum tubes are used for testing in clinical chemistry and immunology, hormones, therapeutic drug monitoring (TDM) and serology.

If anticoagulants are added to the whole blood, it does not clot and the plasma can be obtained through centrifugation. The plasma contains all clotting and fibrinolysis factors in active form. Plasma tubes are also used for determining blood parameters in clinical chemistry.

During centrifugation, the separator gel moves to the boundary surface between the fluid and blood cell portion of the blood, where it forms a stable barrier and separates the remainder from the cell component parts. Blood collection tubes are used for testing in clinical chemistry and immunology, hormones, TDM and serology.

The material of the blood collection tube can, for example, be a thermoplastic such as polyethylene (PE), polypropylene (PP), polystyrene (PS), polyethylene terephthalate (PET), polyethylene furan 2,5-dicarboxylate (PEF), polymethyl methacrylate, polyacrylonitrile, polyamide, acrylnitrile-styrene copolymers and ethylene vinyl alcohol copolymers; or a duroplastic such as unsaturated polyester resins, epoxy resins and epoxy acrylate resins; modified natural resins such as cellulose acetate, cellulose propionate, ethyl cellulose and ethyl chitin; silicates such as soda lime glass, phosphosilicate glass and borosilicate glass, glasses such as quartz glass and combinations thereof, or materials which primarily contain one of the above.

Blood collection tubes according to the invention are preferably made from PET and provide improved safety and hygiene when taking the sample with the advantage that they are transparent.

The blood collection tube can be sealed with a sealing element, preferably a sealing plug.

The interior of the blood collection tube is preferably evacuated and has a sealing plug.

In order to prevent infections, the blood collection tube is sterilized, for instance using electron irradiation, gamma rays or x-rays.

Moreover, further components can be arranged on the inner wall of the blood collection tube. For example, a blood clotting accelerator can be arranged there.

If the blood collection tube is used to obtain blood plasma, the blood collection tube contains an anticoagulant which can also adhere to the inner wall of the blood collection tube. Anticoagulants such as heparin, EDTA or citrate, or other substances known in the art, can be arranged there.

According to a preferred embodiment, 1.4 g of separator gel for the separation of blood serum or blood plasma is filled into a blood collection tube of 100 mm length and 16 mm outer diameter which is subjected to a vacuum, and is then sterilized.

In the method for the separation of blood serum or blood plasma with a blood collection tube according to the present invention, the whole blood is centrifuged in the blood collection tube in order to obtain blood plasma or blood serum in sufficient quantity and quality. The whole blood is collected in the blood collection tube beforehand. Through centrifugation, the separator gel passes between the layer containing the blood cells and the layer containing the blood serum or blood plasma. The density of the blood cells is greater than that of the blood plasma or blood serum and is in a range of approximately 1.06 g/cm³ to 1.11 g/cm³, whereas the density of the blood serum or blood plasma is in a range of 1.025 g/cm³ and 1.030 g/cm³. The density of the separator gel is between that, from 1.038 g/cm³ to 1.058 g/cm³.

The acrylate copolymer for a separator gel for blood collection tubes comprises at least two monomers and a solvent, with ≤50 ppm, in particular ≤20 ppm of a first monomer, in particular n-butyl acrylate, and ≤200 ppm, preferably ≤100 ppm, in particular ≤80 ppm of a second monomer, in particular 2-ethylhexyl acrylate as well as a solvent content, in particular toluene, of ≤1000 ppm, in particular ≤300 ppm.

For the production of the polymer component of the acrylate copolymer, the ratio of the monomers n-butyl acrylate to 2-ethylhexyl acrylate is 2:1 to 9:1, in particular 3:1 to 5:1, preferably 4:1.

For the production of the polymer component at least n-butyl acrylate is used in a quantity of 30 wt. % to 50 wt. %, preferably 35 wt. % to 45 wt. %, in particular 38 wt. % to 42 wt. %, and 2-ethylhexyl acrylate is used in a quantity of 2 wt. % to 20 wt. %, preferably 5 wt. % to 15 wt. %, in particular 8 wt. % to 12 wt. %.

As a solvent for the production of the polymer component an aromatic solvent, preferably toluene and/or xylene, is used in a quantity of 30 wt. % to 70 wt. %, preferably 40 wt. % to 60 wt. %, in particular 45 wt. % to 55 wt. %.

An organic peroxide, preferably 1,1,3,3-tetramethylbutylperoxy-2-ethylhexanoate, is preferably used in a quantity of 0.05 wt. % to 5 wt. %, preferably 0.1 wt. % to 1 wt. %, in particular 0.3 wt. % to 0.6 wt. %, as an initiator of the polymerization of the at least two monomers for the production of the polymer component.

For the production of the polymer component for the production of the acrylate copolymer for the separator gel, preferably approximately 50 wt. % solvent, 40 wt. % n-butyl acrylate and 10 wt. % 2-ethylhexyl acrylate and an initiator in a quantity of less than 1 wt. % are used.

The purified acrylate copolymer has a density of 1.01 g/cm³ to 1.04 g/cm³, preferably 1.025 g/cm³ to 1.035 g/cm³, in particular between 1.030 g/cm³ to 1.033 g/cm³, at 20° C. and a viscosity of 60 Pa·s to 180 Pa·s, preferably 70 Pa·s to 130 Pa·s, in particular 90 Pa·s to 110 Pa·s, at 20° C.

The purified acrylate copolymer according to the invention for the production of a separator gel for a blood collection tube has a residual content of n-butyl acrylate monomer of <50 ppm, in particular <20 ppm, and a residual content of 2-ethylhexyl acrylate monomer of <200 ppm, preferably <100 ppm, in particular <80 ppm.

According to particularly preferred embodiments, the acrylate copolymer has a residual content of <5 ppm of n-butyl acrylate monomer and <20 ppm of 2-ethylhexyl acrylate monomer and at the same time a residual toluene content of <50 ppm.

The acrylate copolymer according to the invention for a separator gel for blood collection tubes is preferably produced through radical solvent polymerization in feed procedure from a polymer solution, wherein for the purification of the acrylate copolymer at least the solvent is separated by multistage distillation, such that a residual solvent content of <1000 ppm, in particular <300 ppm, is achieved.

The starting temperature of the polymerization for the acrylate copolymer is at the boiling point of the respective solvent used. The different monomers are fed over a longer period of at least 60 minutes, preferably 120 minutes, whereby the monomer ratio of NBA to EHA is preferably 4:1. At the same time, the initiator of the polymerization solvent is fed over the same period or longer, in particular up to 240 minutes.

According to an alternative embodiment, the production of the polymer component of the acrylate copolymer for a separator gel for blood collection tubes can also be performed using an azo-based polymerization initiator such as azobisisobutyronitrile (AIBN).

Through multistage distillation, residual monomers are also separated from the polymer solution of the acrylate copolymer, such that ≤50 ppm, in particular <20 ppm of a first monomer, in particular n-butyl acrylate, and <200 ppm, preferably <100 ppm, in particular <80 ppm of a second monomer, in particular 2-ethylhexyl acrylate, are achieved.

The distillation is performed continuously with wiped film rotation evaporators.

The wiped film evaporation of volatile substances, predominantly toluene and/or xylene, but also of the monomers n-butyl acrylate and 2-ethylhexyl acrylate, is performed at a temperature between 130° C. and 150° C. jacket temperature and a pressure of 90 mbar to 100 mbar.

The polymer discharged from the wiped film rotation evaporator is fed into a flash evaporation stage, and where applicable is subjected beforehand to interim heating.

The separation of the solvent and where applicable the residual monomers is performed in a vacuum.

Where applicable, the solvent and where applicable the residual monomers are distilled off to their desired final content in a subsequent short-path evaporation stage.

The short-path evaporation is preferably performed at a temperature of 150° C. jacket temperature at a pressure of less than 0.1 mbar.

The solvent and where applicable the residual monomers are frozen out and subsequently discharged in the downstream cold trap system.

The present invention is described in more detail with reference to the following examples. It is, however, not limited to these examples.

### Production of an Acrylate Copolymer

The acrylate copolymer is produced by means of radical solvent polymerization at normal pressure during boiling at 111° C. in feed procedure. The boiling temperature of the polymer solution increases during the reaction to a maximum of approximately 117° C. Beginning at the same time, the monomers are fed over a period of 120 minutes and the initiator over a period of 240 minutes into the provided boiling solvent in the reactor.

Composition and chemical as well as physical properties of the acrylate copolymer

Table 3 shows different compositions of the acrylate copolymer, which form the basis for the production of the separator gel according to the invention. As components for the production of the different compositions of the acrylate copolymers, toluene was used as solvent, n-butyl acrylate (NBA) and 2-ethylhexyl acrylate (EHA) as monomers, and 1,1,3,3-tetramethylbutylperoxy-2-ethylhexanoate (e.g. Trigonox® 421 from Nouryon (T421) or azobisisobutyronitrile (AIBN)) as initiator.

Table 3 lists the viscosity and the density and the residual contents of the monomers and the toluene solvent, whereby the parameters were determined by means of the measuring methods mentioned above.

TABLE 3

| | NBA:EHA | | Viscosity | Density | Residual content (purity) | | |
|---|---|---|---|---|---|---|---|
| | (Mass | | 20° C. | 20° C. | Toluene | NBA | EHA |
| No. | ratio) | Initiator | [Pa · s] | [g/cm³] | [ppm] | [ppm] | [ppm] |
| 1 | 3:1 | AlBN | 109 | 1.026 ⁽² | 59 | <5 | 158 |
| 2 | 9:1 | T421 | 166 | 1.038 ⁽¹ | 19 | <5 | 60 |
| 3 | 9:1 | T421 | 67 | 1.038 ⁽¹ | 24 | <5 | <5 |
| 4 | 9:1 | T421 | 124 | 1.038 ⁽¹ | 6 | <5 | <5 |
| 5 | 3:1 | T421 | 64 | 1.027 ⁽¹ | 10 | <5 | 6 |
| 6 | 3:1 | T421 | 89 | 1.028 ⁽¹ | <5 | <5 | 24 |
| 7 | 4:1 | T421 | 100 | 1.032 ⁽¹ | 33 | <5 | 13 |
| 8 | 4:1 | T421 | 101 | 1.031 ⁽² | <5 | <5 | <5 |

⁽¹ Measuring method: Gas pycnometer
⁽² Measuring method: Oscillating U-tube density meter

If the measurement results of the GC measurements of the residual content of toluene, NBA or EHA provide results which are not detectable or are smaller than 5 ppm, they are listed in Table 3 as less than 5.

### Composition and Chemical as Well as Physical Properties of the Separator Gel

Table 4 lists the composition, the viscosity and the density of separator gels which were produced using the examples of acrylate copolymers described in Table 3, whereby the parameters were also determined by means of the measuring methods listed above.

TABLE 4

| | | | | | | | | | | Density | | |
|---|---|---|---|---|---|---|---|---|---|---|---|---|
| | | Silica | | Silica | | | Poly- | | | at | Viscosity | |
| | | hydro- | [wt. | hydro- | [wt. | Silicone | alkylene | [wt. | Titanium | 20° C. | at 20° C. | |
| No. | Polymer | philic | %] | phobic | %] | oil | glycol | %] | dioxide | [g/cm³] | [Pa · s] | TI |
| 1 | 2 | Aerosil | 2.75 | — | — | Xiameter ™ | — | 0.15 | 0.0125% | 1.055⁽¹ | N/A | N/A |
| | | 200 | | | | OFX- | | | | | | |
| | | | | | | 0193 | | | | | | |
| 2 | 3 | Aerosil | 2.75 | Aero- | 0.4 | Xiameter ™ | — | 0.15 | — | 1.054⁽¹ | N/A | N/A |
| | | 200 | | sil ®R8200 | | OFX- | | | | | | |
| | | | | | | 0193 | | | | | | |
| 3 | 3 | Aerosil | 2.75 | Aero- | 0.4 | Xiameter ™ | — | 0.15 | — | 1.053⁽¹ | N/A | N/A |
| | | 200 | | sil ®R208 | | OFX- | | | | | | |
| | | | | | | 0193 | | | | | | |
| 4 | 4 | Aerosil | 2.75 | — | — | Xiameter ™ | — | 0.15 | — | 1.054⁽¹ | N/A | N/A |
| | | 200 | | | | OFX- | | | | | | |
| | | | | | | 0190 | | | | | | |
| 5 | 4 | Aerosil | 2.75 | Aero- | 0.4 | Xiameter ™ | — | 0.15 | — | 1.055⁽¹ | N/A | N/A |
| | | 200 | | sil ®R974 | | OFX- | | | | | | |
| | | | | | | 0193 | | | | | | |
| 6 | 5 | Aerosil | 2.75 | — | — | Xiameter ™ | — | 0.15 | 0.0125% | 1.043⁽¹ | N/A | N/A |
| | | 200 | | | | OFX- | | | | | | |
| | | | | | | 0190 | | | | | | |
| 7 | 6 | Aerosil | 2.65 | Aero- | 1.0 | Xiameter ™ | — | 0.15 | 0.0125% | 1.048⁽¹ | N/A | N/A |
| | | 200 | | sil ®R974 | | OFX- | | | | | | |
| | | | | | | 0190 | | | | | | |
| 8 | 7 | Aerosil | 2.75 | — | — | Xiameter ™ | — | 0.50 | 0.0125% | 1.047⁽¹ | N/A | N/A |
| | | 200 | | | | OFX- | | | | | | |
| | | | | | | 0190 | | | | | | |
| 9 | 8 | Aerosil | 2.75 | — | — | Xiameter ™ | — | 0.15 | 0.0125% | 1.047⁽² | 230 | 1.4 |
| | | 200 | | | | OFX- | | | | | | |
| | | | | | | 0190 | | | | | | |
| 10 | 9 | Aerosil | 2.75 | — | — | — | PPG | 0.15 | 0.0125% | 1.047⁽² | 214 | 1.3 |
| | | 200 | | | | | (Mn 624) | | | | | |
| 11 | 9 | Aerosil | 2.75 | — | — | — | PPG | 0.135 | 0.0125% | 1.047⁽² | 211 | 1.3 |
| | | 200 | | | | | (Mn 400) | | | | | |
| | | | | | | | PEG | 0.015 | | | | |
| | | | | | | | (Mn 400) | | | | | |

⁽¹Measuring method: Gas pycnometer
⁽²Measuring method: Oscillating U-tube density meter
AEROSIL ® Product line of EVONIK Industries AG
XIAMETER ™ Product line of DOW Corning
Titanium dioxide KRONOS 1171

All separator gels listed above showed a residual content of 10 ppm to 50 ppm of toluene and ≤20 ppm of n-butyl acrylate and ≤80 ppm of 2-ethylhexyl acrylate. The measurement values of the residual contents of solvent and monomer of the acrylate copolymers used for the production of the separator gel also apply to the separator gels, as there is no further addition of monomers or solvents during the mixing process of the components for the production of the separator gel.

According to a preferred embodiment variation, the separator gel has a composition of 97.1 wt. % purified acrylate copolymer, 2.75 wt. % silica, 0.15 wt. % silicone oil and 0.0125 wt. % titanium dioxide.

According to a further embodiment variation, the separator gel has a composition of 97.1 wt. % purified acrylate copolymer, 2.75 wt. % silica, 0.15 wt. % PEG and/or PPG and 0.0125 wt. % titanium dioxide.

A possible further embodiment variation of the separator gel comprises an acrylate copolymer, silica and a silicone oil and at least one polyalkylene glycol. This embodiment preferably consists of 97.1 wt. % purified acrylate copolymer, 2.75 wt. % silica and a total quantity of 0.15 wt. % silicone oil, PEG and PPG as well as 0.0125 wt. % titanium dioxide.

In a study, on the one hand blood collection tubes for obtaining serum and on the other hand blood collection tubes for obtaining plasma with the separator gel according to the invention (A, D) and with an established separator gel approved on the market (H, J) were compared. To do so, blood was taken from 20 test persons with different blood collection tubes and then tested. The mean values of the specific analytes in the blood collection tubes with the separator gel according to the invention were compared with the mean values of the specific analytes in the blood collection tubes with the separator gel approved on the market of the same 20 test persons.

The following Table 5 shows the measurement results for the blood collection tubes for obtaining serum.

TABLE 5

| | | A | H | | |
|---|---|---|---|---|---|
| | | Mean | Mean | | |
| Parameter | Group | value | value | Criterion % | Equivalence |
| Sodium | Electrolyte | 138.85 | 138.70 | 3.00 | Yes |
| Potassium | Electrolyte | 4.09 | 4.07 | 4.50 | Yes |
| Chloride | Electrolyte | 104.25 | 104.25 | 4.50 | Yes |
| Total protein | Proteins | 68.12 | 67.18 | 6.00 | Yes |
| Albumin | Proteins | 43.51 | 43.56 | 8.00 | Yes |
| Bilirubin | Liver function | 0.68 | 0.68 | 20.00 | Yes |
| Alanin | Liver function | 25.60 | 25.60 | 11.50 | Yes |
| aminotransferase | | | | | |
| Glucose | General tests | 95.40 | 95.45 | 8.00 | Yes |
| Calcium | General tests | 2.39 | 2.39 | 6.00 | Yes |
| Creatinine | Kidney function | 0.84 | 0.84 | 11.00 | Yes |

Table 6 shows the results of the analytes from blood collection tubes for obtaining plasma.

TABLE 6

| | | D | J | | |
|---|---|---|---|---|---|
| | | Mean | Mean | | |
| Parameter | Group | value | value | Criterion % | Equivalence |
| Sodium | Electrolyte | 138.75 | 138.55 | 3.00 | Yes |
| Potassium | Electrolyte | 3.79 | 3.80 | 4.50 | Yes |
| Chloride | Electrolyte | 104.40 | 104.45 | 4.50 | Yes |
| Total protein | Proteins | 69.60 | 69.36 | 6.00 | Yes |
| Albumin | Proteins | 43.16 | 43.13 | 8.00 | Yes |
| Bilirubin | Liver function | 0.68 | 0.68 | 20.00 | Yes |
| Alanin | Liver function | 25.55 | 25.00 | 11.50 | Yes |
| aminotransferase | | | | | |
| Glucose | General tests | 96.05 | 97.20 | 8.00 | Yes |
| Calcium | General tests | 2.37 | 2.37 | 6.00 | Yes |
| Creatinine | Kidney function | 0.84 | 0.83 | 11.00 | Yes |

Based on the comprehensive metabolic panel, one or more parameters were selected per group (general tests, electrolytes, proteins, liver function, kidney function) for the study.

In the first step, the selected parameters monitor the state of health and provide an overall impression of the body's chemical balance and the metabolism, both in routine examination as well as in in-patient or out-patient examination.

The common acceptance criteria (for example RILIBAEK (Guideline of the German Medical Association on Quality Assurance in Medical Laboratory Examinations), CLIR) are listed in the Criterion % column. Equivalence between the blood collection tubes with the separator gel according to the invention and the separator gel known in the prior art was determined if the percentage difference of the two mean values is below the acceptance criterion.

No clinically significant differences could be identified in the study between the blood collection tubes with the separator gel according to the invention and the established separator gel approved on the market.

The example embodiments show possible embodiment variations, although it is to be noted here that the invention is not limited to the specifically represented embodiment variations of the same, but rather various combinations of the individual embodiment variations with one another are possible, and that given the technical teachings provided by the present invention this variation possibility is within the ability of the skilled person in this technical field.

The scope of protection is defined by the claims. The description and the drawings should, however, be consulted when construing the claims. Individual features or combinations of features from the various example embodiments as shown and described can constitute separate inventive solutions. The problem to be solved by the individual inventive solutions can be derived from the description.

All value ranges specified in the current description are to be understood such that they include any and all sub-ranges, e.g., the specification 1 to 10 is to be understood such that all sub-ranges, starting from the lower limit 1 and the upper limit 10 are included, i.e., all sub-ranges begin with a lower limit of 1 or more and end at an upper limit of 10 or less, e.g., 1 to 1.7, or 3.2 to 8.1, or 5.5 to 10.

As a matter of form and by way of conclusion, it is noted that, to improve understanding of the structure, elements have partially not been shown to scale and/or enlarged and/or shrunk.

It is to be understood that the above description is intended to be illustrative and not restrictive. Many applications other than the examples provided would be upon reading the above description. The scope of the disclosure should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the disclosure is capable of modification and variation and is limited only by the following claims.

All terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary.

It should be understood that references to a single element are not necessarily so limited and may include one or more of such element. Any directional references (e.g., plus, minus, upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of examples/embodiments.

It will also be understood that, although the terms first, second, etc. are, in some instances, used herein to describe various elements, these elements should not be limited by these terms. These terms are only used to distinguish one element from another. For example, a first element could be termed a second element, and, similarly, a second element could be termed a first element, without departing from the scope of the various described embodiments. The first element and the second element are both element, but they are not the same element.

The terminology used in the description of the various described embodiments herein is for the purpose of describing particular embodiments only and is not intended to be limiting. As used in the description of the various described embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

1. A separator gel for blood collection tubes for separation of blood serum or blood plasma from blood cells, comprising: an acrylate copolymer, silica and silicone oil and/or at least one polyalkylene glycol, wherein the separator gel contains ≤1000 ppm of solvent.

2. The separator gel according to claim 1, wherein for polymerization of the acrylate copolymer for production of a polymer component at least two monomers, a solvent, and an initiator are used.

3. The separator gel according to claim 1, wherein for production of a polymer component of the acryulate copolymer, monomers including n-butyl acrylate and 2-ethylhexyl acrylate are used, wherein a monomer ratio of n-butyl acrylate to 2-ethylhexyl acrylate is 2:1 to 9:1.

4. The separator gel according to claim 1, wherein the acrylate copolymer contains ≤1000 ppm of solvent.

5. The separator gel according to claim 1, wherein the acrylate copolymer has a residual content of n-butyl acrylate monomer of ≤50 ppm.

6. The separator gel according to claim 1, wherein the acrylate copolymer has a residual content of 2-ethylhexyl acrylate monomer of ≤200 ppm.

7. A method for producing a separator gel for blood collection tubes for the separation of blood serum or blood plasma, comprising an acrylate copolymer, silica and silicone oil and/or at least one polyalkylene glycol, the method including mixing the acrylate copolymer, silica and silicone oil and/or at least one polyalkylene glycol.

8. The method for producing the separator gel according to claim 7, wherein for producing a polymer component of the acrylate copolymer at least n-butyl acrylate is used in a quantity of 30 wt. % to 50 wt. %, and 2-ethylhexyl acrylate is used in a quantity of 2 wt. % to 20 wt. %.

9. The method for producing the separator gel according to claim 7, a solvent for producing a polymer component of the acrylate copolymer an aromatic solvent is used.

10. The method for producing the separator gel according to claim 9, wherein the aromatic solvent comprises toluene and/or xylene.

11. The method for producing the separator gel according to claim 9, wherein the aromatic solvent is used in a quantity of 30 wt. % to 70 wt. %.

12. The method for producing the separator gel according to claim 7, wherein as an initiator for polymerization of the at least two monomers an organic peroxide is used for the production of the polymer component.

13. The method for producing the separator gel according to claim 12, wherein the organic peroxide comprises 1,1,3,3-tetramethylbutylperoxy-2-ethylhexanoate.

14. The method for producing the separator gel according to claim 12, wherein the organic peroxide is used in a quantity of 0.05 wt. % to 5 wt. %.

15. The method for producing the separator gel according to claim 7, further comprising mixing in titanium dioxide.

16. A blood collection tube for the separation of blood serum or blood plasma from blood cells with a separator gel according to claim 1.

17. A method for producing an acrylate copolymer for a separator gel for blood collection tubes, comprising radical solvent polymerization in feed procedure from a polymer component, wherein for purification of the acrylate copolymer, at least one solvent is separated by multistage distillation, such that a residual solvent quantity of ≤1000 ppm is achieved.

18. The method according to claim 17, wherein the multistage distillation is performed as wiped film distillation and whereby residual monomers are separated from the polymer component of the acrylate copolymer, such that ≤50 ppm of a first monomer and ≤200 ppm of a second monomer are achieved.

19. The method according to claim 18, wherein the first monomer is n-butyl acrylate and the second monomer is 2-ethylhexyl acrylate.

20. The method according to claim 17, wherein in a subsequent short-path evaporation stage, the solvent and where applicable the residual monomers are distilled off to their final content.
